(19) United States
(12) Patent Application Publication
(10) Pub. No.: US 2017/0049599 A1
(43) Pub. Date: Feb. 23, 2017

(54) SYSTEMS AND METHODS FOR ORTHOPEDIC SUPPORTS

(71) Applicant: Therapeutic Envisions, Inc., Libertyville, IL (US)

(72) Inventor: Charles Edward Hodges, IV, Libertyville, IL (US)

(73) Assignee: Therapeutic Envisions, Inc., Libertyville, IL (US)

(21) Appl. No.: 15/242,139

(22) Filed: Aug. 19, 2016

Related U.S. Application Data
(60) Provisional application No. 62/207,248, filed on Aug. 19, 2015.

(51) Int. Cl. A61F 5/01; A41D 13/12; A41B 1/00; A41D 1/06; A61F 5/03; A61F 5/02

(52) U.S. Cl. CPC A61F 5/0106; A61F 5/03; A61F 5/02; A41B 1/00; A41D 1/06; A41D 13/1263; A61B 2562/0219

(57) ABSTRACT

A garment for providing orthopedic support or therapy to a human body may include a tubular flexible element and a tension strap. The tubular flexible element may be disposed around at least some portion of a human body and include flexible and a touch fastener portions. The flexible portion may be at an exterior of the tubular flexible element and cover a first portion of the body. The touch fastener portion may be at the exterior of the tubular flexible element and cover a second portion of the body. The tension strap may include first and second ends, where the first end includes a first touch fastener to be coupled with the touch fastener portion at a first location, and the second end includes a second touch fastener to be coupled with the touch fastener portion at a second location, thereby applying tension between the first and second locations.

### CROSS-REFERENCE TO RELATED APPLICATIONS

This application claims priority to Provisional U.S. Patent Application No. 62/207,248 filed Aug. 19, 2015, entitled “SYSTEMS AND METHODS FOR ORTHOPEDIC SUPPORTS,” the entire disclosure of which is hereby incorporated by reference, for all purposes, as if fully set forth herein.

### BACKGROUND OF THE INVENTION

Embodiments of the invention relate generally to protective and/or therapeutic orthopedic wear. More specifically, embodiments of the invention relate to clothing which allows for customized application of protective and/or therapeutic elements to targeted areas of the body.

Current orthopedic support systems tend to be generic, sized for a variety of human beings, and designed to address a variety of orthopedic issues. Some current systems may support or protect a group of joints or muscles during athletic, occupational, or everyday activities. Other current systems may provide therapeutic value to a particular area of the body, and in the best cases possibly be provided for two to three size ranges of individuals.

This one, two, or three-size fits all approach, to both the individual concerned, and the joint or muscle area involved does not adequately take into account the almost infinite anatomic variation of individuals. Likewise, injuries or particular orthopedic needs of an individual user can also be infinitely varied. Current orthopedic garments are usually only readily available to address the most common of injuries and needs. Customization for the particular needs/injury of specific individuals are largely unaddressed unless the need/injury is very typical and the specific individual is of common shape and size.

Embodiments of the invention provide solutions to these and other problems in the prior art.

### BRIEF DESCRIPTION OF THE INVENTION

In one embodiment, a garment for providing orthopedic support or therapy to a human body is provided. The garment may include a tubular flexible element and a tension strap. The tubular flexible element may be configured to be disposed around at least some portion of a human body and include a flexible portion and a touch fastener portion. The flexible portion may be at an exterior of the tubular flexible element and cover a first portion of the human body. The touch fastener portion may be at the exterior of the tubular flexible element and cover a second portion of the human body. The tension strap may include a first end and a second end, where the first end includes a first touch fastener configured to be coupled with the touch fastener portion at a first location, and the second end includes a second touch fastener configured to be coupled with the touch fastener portion at a second location, thereby applying tension between the first location and the second location.

### BRIEF DESCRIPTION OF THE DRAWINGS

The present invention is described in conjunction with the appended figures:

FIG. 1 is a schematic view of one garment of the invention, particularly a pair of pants, which provides means for orthopedic support and therapy to be applied to a wearer;

FIG. 2A is a schematic view of another garment of the invention, particularly a shirt, which provides means for orthopedic support and therapy to be applied to a wearer;

FIG. 2B is a schematic view of another garment of the invention, particularly a tubular sleeve, which provides means for orthopedic support and therapy to be applied to a wearer;

FIG. 2C is a schematic view of another garment of the invention, particularly a bodysuit, which provides means for orthopedic support and therapy to be applied to a wearer;

FIG. 3 is a schematic view of the garment from FIG. 1 where the tension strap has been applied to the tubular flexible element;

FIG. 4 is a schematic view of an alternative tension strap of the invention having electronics included therewith; and

FIG. 5 is a schematic view of an alternative tensions strap of the invention having a detachable electronics package.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

For example, any detail discussed with regard to one embodiment may or may not be present in all contemplated versions of that embodiment. Likewise, any detail discussed with regard to one embodiment may or may not be present in all contemplated versions of other embodiments discussed herein. Finally, the absence of discussion of any detail with regard to embodiment herein shall be an implicit recognition that such detail may or may not be present in any version of any embodiment discussed herein.

Specific details are given in the following description to provide a thorough understanding of the embodiments. However, it will be understood by one of ordinary skill in the art that the embodiments may be practiced without these specific details. For example, circuits, systems, networks, processes, and other elements in the invention may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Furthermore, embodiments of the invention may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware, or microcode, the program code or code segments to perform the necessary tasks may be stored in a machine readable medium and a processor or processors may perform the necessary tasks.

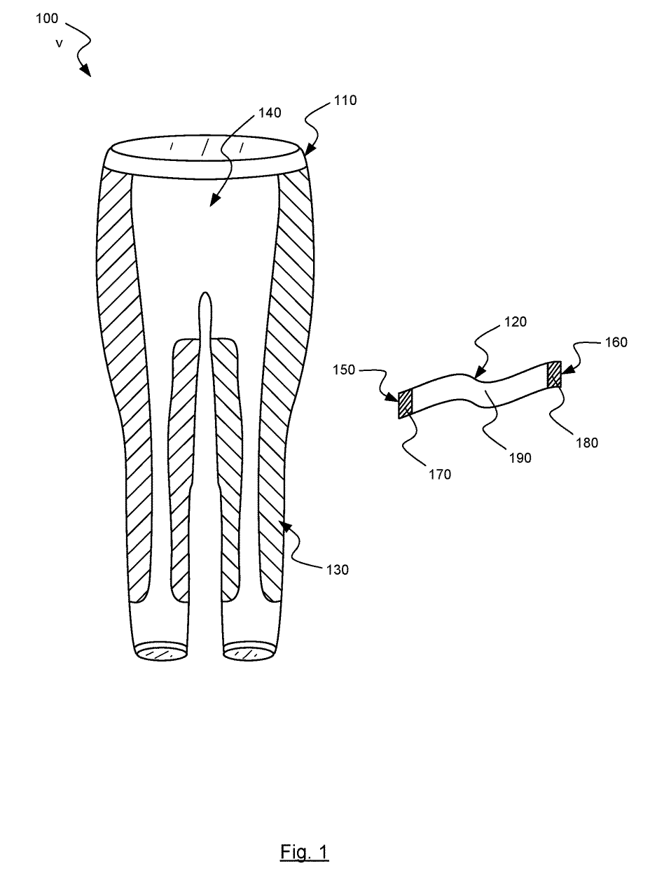

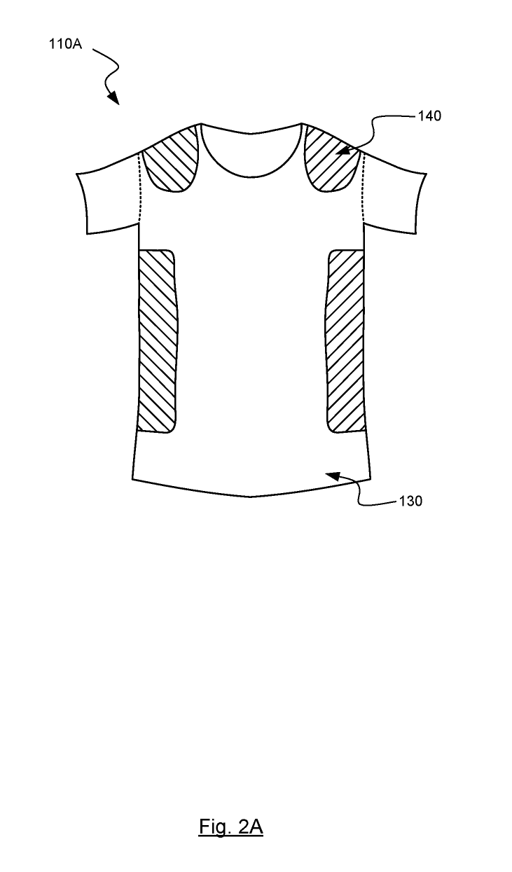

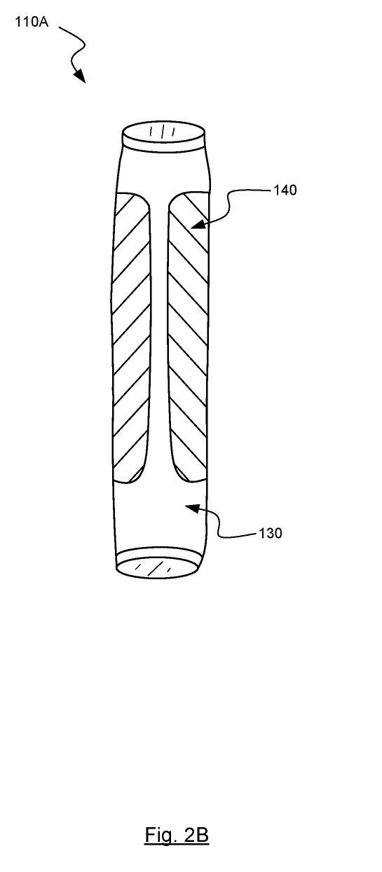

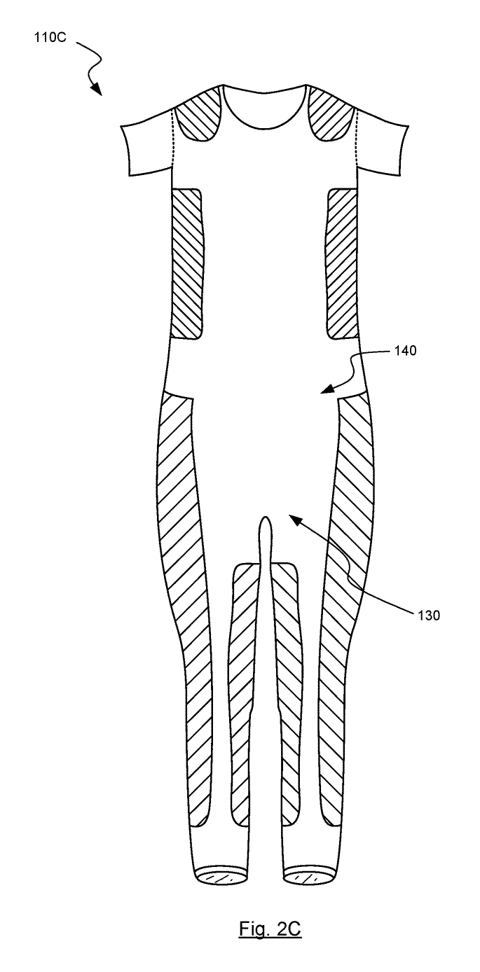

Turning to FIG. 1, in one embodiment of the invention, a garment 100 for providing orthopedic support or therapy to a human body is provided. Garment 100 may include a tubular flexible element 110 and a tension strap 120. Though tubular flexible element 110 is shown in this embodiment as a pair of pants, in other embodiments, as shown in FIGS. 2A-C, tubular flexible element 110 could be a sleeved or sleeveless shirt 110A; a tubular sleeve 110B for an arm, leg, or other joint area; or a body suit 110C. Tubular sleeve 110B, or other shaped garments, may allow for the garment to be used at various joints or locations on the body. Merely by way of example, tubular sleeve may be worn by a user around their elbow, shin/calf, Achilles tendon, ankle, shoulder, wrist, neck, and in larger versions around some portion of the back, hip, torso, sternum, waist, thighs, etc. Other shapes of garments are also possible within the scope of this disclosure.

Tubular flexible element 110 may be configured to be disposed around at least some portion of a human body and include a flexible portion 130 and a touch fastener portion 140. Flexible portion 130 may be at an exterior of tubular flexible element 110 and cover a first portion of the human body. Touch fastener portion 140 may also be at the exterior of tubular flexible element 110 and cover a second portion of the human body. In other words, portions of the exterior of tubular flexible element 110 will consist of flexible portion 130, while remaining portions of the exterior consist of touch fastener portion 140. In some embodiments, flexible portion 130 and/or material thereof, may also underlie touch fastener portion 140. In other embodiments, a different material may underlie touch fastener portion 140.

Flexible portion 130 may include neoprene and/or other flexible materials such as polyesters, nylon, lycra, spandex, rayon, etc. which are flexible. In some embodiments, flexibility may be multidirectional, while in other embodiments, flexibility may be unidirectional. In yet other embodiments, combinations of the same will be present, either through the use of a consistent material, or combinations of materials.

Touch fastener portion 140 may include Dual Lock™, Velcro™ or other hook and loop material, and/or any other touch fastener material which allows for two materials to be coupled to each other by pressing or applying them against each other. Touch fastener portion 140 may consist of the loop element of the hook and loop or hook and loop-like material so as not to unintentionally attract fabric to attach to the garment.

Tension strap 120 may include a first end 150 and a second end 160. First end 150 may include a first touch fastener 170 configured to be coupled with touch fastener portion 130 at a first location (for example, anywhere within touch fastener portion 140). In the embodiment described, first touch fastener 170 may be the hook element of hook and loop (or the like) material, so as to mate with a loop element of touch fastener portion 140. Second end 160 may include a second touch fastener 180 configured to be coupled with touch fastener portion 140 at a second location (for example, a different location than where first touch fastener 170 is coupled with touch fastener portion 140), thereby applying tension between the first location and the second location, or at least limiting the movement of the two locations of tubular flexible element 110 away from each other if the limit of the elasticity of tension strap 120 is reached. Thus for example, if a particular tensions strap 120 is of fixed length (non-flexible), tension will be applied by the strap when movement of the two locations attempts to exceed the fixed length of tensions strap 120.

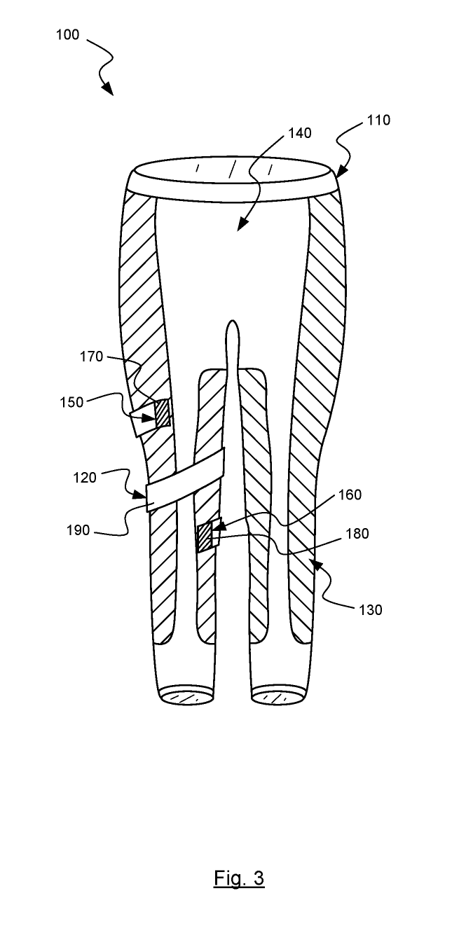

Merely by way of example, one example positioning of tension strap 120 on tubular flexible element is shown in FIG. 3, where tension strap 120 has been coupled at one end below the knee of the pants, wrapped around the leg of the pants, and then coupled at the other end above the knee.

Though FIGS. 1 and 2A-C show flexible portion 130 and touch fastener portion 140 in certain specific areas of the particular garment therein, one of skill in the art will now understand, based on the contents of this disclosure, that flexible portion 130 and touch fastener portion 140 could be arranged differently than shown. In some embodiments flexible portion may take up more or less of a garment than shown, and be provided in different shaped areas thereon. Likewise, in these or other embodiments, touch fastener portion 140 may take up more or less of a garment than shown, and be provided in different shaped areas thereon. Ideally, the garment will remain flexible because of the presence of adequate flexible portion 130 areas, but provide touch fastener portion 140 in as many areas as necessary to provide means to configure tension strap 120 in any manner desired, thereby providing maximum customized configurations for different sized users and different therapeutic/protective needs.

In the embodiment shown in FIG. 3, second touch fastener 180 may be the hook element of hook and loop material, so as to mate with a loop element of touch fastener portion 140. In various embodiments, first and second touch fasteners 170, 180 may be located on one or both sides of tension strap 120. In some embodiments, one side of tension strap 120 may have hook elements of a hook and loop material at first and second touch fasteners 170, 180, while the other side of tensions strap may have loop elements of a hook and loop material, thereby allowing additional elements (e.g., tension straps or other elements) to be coupled with the outside portion of first and second touch fasteners 170, 180.

Tension strap 120 may also include an intermediate portion 190 between first end 150 and second end 160 by which the construction characteristics thereof change the overall elasticity characteristics of tension strap 120. Merely by way of example, intermediate portion 190 could be constructed of an inelastic material, thereby setting a maximum length of tension strap 120. This may be referred to herein as a fixed length assembly. Alternatively, intermediate portion 190 could be constructed of an elastic material, thereby setting a default length of tension strap 120 (an un-stretched length), but allowing for a maximum potential length greater than the default length (a fully stretched length). This may be referred to herein as a variable length assembly. In such an embodiment, the spring rate of the elastic material used in intermediate portion 190 could be varied to achieve different strengths of feedback on the user when tension strap 120 is stretched between its coupling points on touch fastener portion 140.

In these or other embodiments, intermediate portion 190 may also include a third touch fastener on the same and/or different side than the first and second touch fastener 170, 180 elements which couple with the touch fastener portion 140. Such a third touch fastener may only be present in a certain location or locations on the underside of intermediate portion 190. In such embodiments, when tension strap 120 is coupled with garment 100, the tension between first touch fastener 170 and third touch fastener could be varied from the tension provided between third touch fastener and second touch fastener 190. Thus, for example, the spring rate in the construction between first touch fastener 170 and third touch fastener could be higher or lower than the spring rate between third touch fastener and second touch fastener 190.

Alternatively or in addition to the above, having a third touch fastener on an exterior of tension strap 120 may allow for additional tension straps to be coupled onto the top of tensions strap 120. Any number of tensions straps 120 may be present in a given embodiment, either at the same, different, or overlapping locations depending on the locations in which the user desires to have additional orthopedic support. Also, while tension strap 120 is shown as being a constant width along its length, in some embodiments the width of tension strap 120 may vary along the length, including being wider at one end than the other in order to provide more tension across a greater area as necessary in a particular therapeutic scenario.

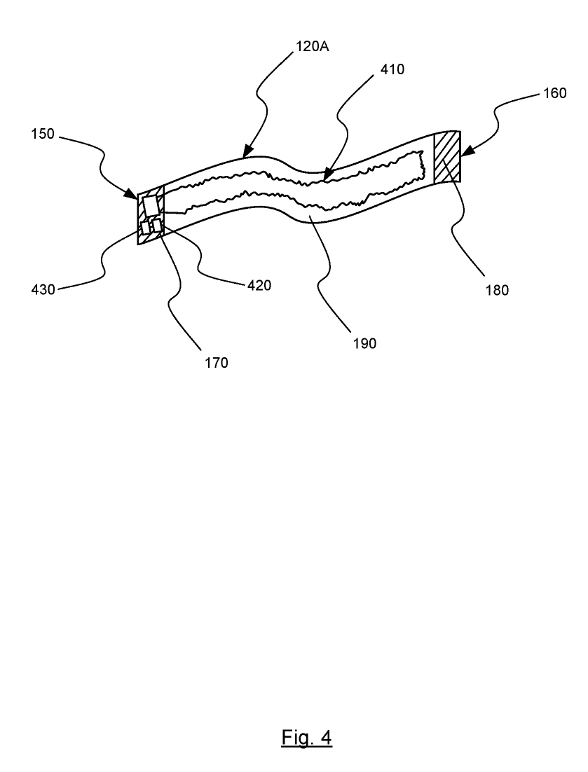

In some embodiments, as shown in FIG. 4, tension strap 120A may include a tension detector 410 disposed on or within intermediate portion 190 for measuring tension between first end 150 and second end 160. An accelerometer 420, gyroscope, and/or other movement detecting device may also be included for determining acceleration, speed, and/or direction of movement of tension strap 120A, and thus acceleration/movement of that particular location on the wearer of garment 100. A communication device 430, employing Bluetooth, WiFi, or other wireless communication medium, may allow for transmission of information from tension detector 410, accelerometer 420, gyroscope, or other movement detecting device to a remote processor/storage device for analysis. Wired communication methods may also be available to communicate data to a remote storage unit and/or processor, perhaps after the data is temporarily stored on a device within tensions strap 120A.

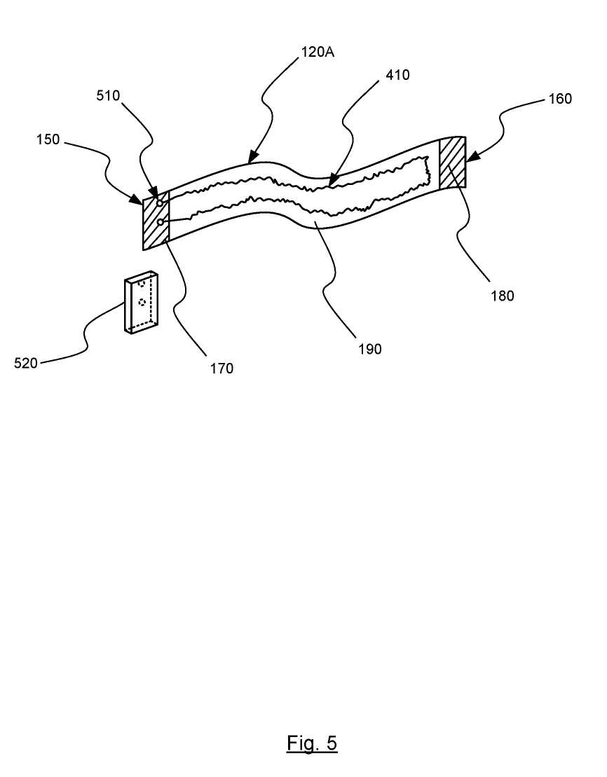

In another similar embodiment, as shown in FIG. 5, tension strap 120B may also include electrical leads 510 which allows for a detachable electronics package 520 to be connected with tension strap 120B. Detachable electronics package 520 may include at least a portion of a tension detector 410 for measuring tension between the first end and the second end, as well as an accelerometer 420, gyroscope, and/or other movement detecting device. A communication device may also be present for either wired or wireless communication to a remote storage unit and/or processor for analysis of the data.

In some embodiments, a compression detector may also be present in tubular flexible element 110, or more commonly, in tension strap 120. The compression detector may allow for determining the amount of compression being applied to the user at a given point on tubular flexible element 110 or tension strap 120. Data from the compression detector may be passed on to remote storage/processors through communication means as described above with regard to tension detector 410, accelerometer 420, gyroscope, and/or other movement detecting device.

In addition to the aforementioned features, garment 100 may also include and feature described by co-pending U.S. patent application Ser. No. 14/829,867, entitled, “SYSTEMS AND METHODS FOR INCREASING THE EFFECTIVENESS OF A MECHANICAL JOINT BRACE,” the entire disclosure of which is hereby incorporated by reference, for all purposes, as if fully set forth herein. Furthermore, any feature described therein may be detachable from garment 100 via touch fastening portion 130.

Likewise, any feature mentioned in co-pending U.S. patent application Ser. No. 15/167,548, entitled “SYSTEMS AND METHODS FOR THERAPEUTIC STIMULATION VIA GARMENTS AND INSERTS PROVIDED THEREON,” may also be included in garment 100. The entire disclosure of the aforementioned application is hereby incorporated by reference, for all purposes, as if fully set forth herein. Any feature described therein may also be detachable from garment 100 via touch fastening portion 130.

The invention has now been described in detail for the purposes of clarity and understanding. However, it will be appreciated that certain changes and modifications may be practiced within the scope of the appended claims.

What is claimed is:

1. A garment for providing orthopedic support or therapy to a human body, wherein the garment comprises:
a tubular flexible element configured to be disposed around at least some portion of a human body, wherein the tubular flexible element comprises:
a flexible portion, at an exterior of the tubular flexible element, covering a first portion of the human body; and
a touch fastener portion, at the exterior of the tubular flexible element, covering a second portion of the human body; and
a tension strap comprising a first end and a second end, wherein:
the first end comprises a first touch fastener configured to be coupled with the touch fastener portion at a first location;
the second end comprises a second touch fastener configured to be coupled with the touch fastener portion at a second location, thereby applying tension between the first location and the second location.

2. The garment for providing orthopedic support or therapy to a human body of claim 1, wherein the tubular flexible element is a selection from a group consisting of:
a pair of pants;
a sleeved or sleeveless shirt;
a tubular sleeve for an arm, leg, or other joint area; and
a body suit.

3. The garment for providing orthopedic support or therapy to a human body of claim 1, wherein the flexible portion comprises:
neoprene.

4. The garment for providing orthopedic support or therapy to a human body of claim 1, wherein:
the touch fastener portion comprises a loop material of a hook and loop fastening system;
the first touch fastener comprises a hook material of the hook and loop fastening system; and
the second touch fastener comprises the hook material of the hook and loop fastening system.

5. The garment for providing orthopedic support or therapy to a human body of claim 1, wherein the tension strap comprises:
an intermediate portion between the first end and the second end.

6. The garment for providing orthopedic support or therapy to a human body of claim 5, wherein the intermediate portion is selected from a group consisting of:
a fixed length assembly;
a variable length assembly; and
a third touch fastener.

7. The garment for providing orthopedic support or therapy to a human body of claim 5, wherein the intermediate portion comprises a third touch fastener on a side of the tensions strap opposite the first touch fastener and the second touch fastener.

8. The garment for providing orthopedic support or therapy to a human body of claim 5, wherein the intermediate portion comprises a third touch fastener on a side of the tensions strap on which the first touch fastener and the second touch fastener are located.

9. The garment for providing orthopedic support or therapy to a human body of claim 1, wherein:
the tension strap is wider at the first end than at the second end.

10. The garment for providing orthopedic support or therapy to a human body of claim 1, wherein the tension strap comprises:
a tension detector for measuring tension between the first end and the second end; and
a communication device for transmitting information from the tension detector to a processor remote from the garment.

11. The garment for providing orthopedic support or therapy to a human body of claim 1, wherein the tension strap comprises:
a gyroscope and/or an accelerometer; and
a communication device for transmitting information from the gyroscope and/or the accelerometer to a processor remote from the garment.

12. The garment for providing orthopedic support or therapy to a human body of claim 1, wherein the tension strap comprises:
electrical leads configured to couple external devices to electrical components included in the tension strap.

13. The garment for providing orthopedic support or therapy to a human body of claim 12, wherein the tension strap comprises:
a detachable electronics package configured to couple with the electrical leads.

14. The garment for providing orthopedic support or therapy to a human body of claim 13, wherein the detachable electronics package comprises at least one selection from a group consisting of:
at least a portion of a tension detector for measuring tension between the first end and the second end;
a gyroscope and/or an accelerometer; and
a communication device for transmitting information from at least one of the tension detector, the gyroscope, or the accelerometer to a processor remote from the garment.
